(19) United States
(12) Patent Application Publication
(10) Pub. No.: US 2010/0129363 A1
(43) Pub. Date: May 27, 2010

(54) METHODS AND COMPOSITIONS USING PDE4 INHIBITORS FOR THE TREATMENT AND MANAGEMENT OF CANCERS

(76) Inventors: Jerome B. Zeldis, Princeton, NJ (US); Peter H. Schafer, Somerset, NJ (US)

Correspondence Address: JONES DAY, 222 E. 41ST. STREET, NEW YORK, NY, 10017, US

(21) Appl. No.: 12/621,445

(22) Filed: Nov. 18, 2009

Related U.S. Application Data
(63) Continuation-in-part of application No. 10/515,270, filed on May 23, 2005.
(60) Provisional application No. 60/380,842, filed on May 17, 2002.
(60) Provisional application No. 60/424,601, filed on Nov. 6, 2002.

(51) Int. Cl. A61K 39/395; A61K 31/40; A61P 35/02

(52) U.S. Cl. 424/133.1; 514/416

(57) ABSTRACT

Methods of treating, preventing and/or managing hematological cancers are disclosed. Specific methods encompass the administration of a PDE4 inhibitor alone or in combination with a second active agent. The invention further relates to methods of treating leukemias and lymphomas which comprise the administration of a PDE4 inhibitor. Pharmaceutical compositions, single unit dosage forms, and kits suitable for use in methods of the invention are also disclosed.

This application is a continuation-in-part of U.S. patent application Ser. No. 10/515,270, filed May 23, 2005, which is a 371 of PCT/US2003/15468, filed May 16, 2003, the entireties of which are incorporated herein by reference. This application claims the benefit of U.S. provisional application Nos. 60/380,842, filed May 17, 2002, and 60/424,601, filed Nov. 6, 2002, the entireties of which are incorporated herein by reference.

### 1. FIELD OF THE INVENTION

This invention relates to methods of treating, preventing and/or managing specific cancers by the administration of phosphodiesterase 4 (PDE4) inhibitors, alone or in combination with other therapeutics. Specifically, this invention encompasses methods of treating, preventing or managing leukemias, including but not limited to, chronic lymphocytic leukemia and acute lymphoblastic leukemia; and lymphomas, including but not limited to diffuse large B-cell lymphoma using cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide; (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide; (S)—N-{2-[1-(3-ethoxy-4-methoxy-phenyl)-2-methanesulfonylethyl]-1,3-dioxo-2,3-dihydro-1H-isoindol-4-yl}acetamide; and/or 3-(3-acetoamidophthalimido)-3-(3-ethoxy-4-methoxyphenyl)-N-hydroxypropionamide.

The invention also encompasses the use of specific combinations, or “cocktails,” of drugs and other therapy, e.g., radiation to treat these specific cancers, including those refractory to conventional therapy. The invention also relates to pharmaceutical compositions and dosing regimens which comprise a PDE4 inhibitor.

### 2. BACKGROUND OF THE INVENTION

### 2.1 Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples includes cancer of the lung, colon, rectum, prostate, breast, brain, and intestine.

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17^{th} ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or acute myeloblastic leukemia (AML), occurs at all ages and is the more common acute leukemia among adults; it is the form usually associated with irradiation as a causative agent.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000 μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000 μL, but may reach 1,000,000 μL. CML is relatively easy to diagnose because of the presence of the Philadelphia chromosome.

In addition to the acute and chronic categorization, neoplasms are also categorized based upon the cells giving rise to such disorder into precursor or peripheral. See e.g., U.S. patent publication no. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

Lymphoma refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body. Lymphocytic leukemias originate and are most prominent in the marrow and spill over into the blood. They occasionally spread to involve the lymph nodes.

The treatment of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous T-cell lymphoma, cutaneous B-cell lymphoma, diffuse large B-cell lymphoma and low grade follicular lymphoma. Because PDE4 is expressed in lymphoid cells, PDE4 inhibitors have been identified as a target for the treatment of lymphomas. See, e.g., Smith et al., Blood, 2005, 105: 308-316.

Diffuse large B-cell lymphoma (DLBCL) accounts for approximately one-third of non-Hodgkin's lymphomas. While some DLBCL patients are cured with traditional chemotherapy, the remainder die from the disease. PDE4B expression has been shown to correlate with treatment-resistant DLBCL and relapse in DLBCL patients treated with traditional chemotherapy regimens. Smith et al., Blood, 2005, 105: 308-316. Thus, PDE4 inhibitors may be useful for the treatment of DLBCL, particulary in refractory cases.

Phophodiesterase (PDE) activity has long been associated with haematological malignancies such as leukemias. See, e.g., Lerner & Epstein, Biochem. J., 2006, 393: 21-42; Epstein et al., Cancer Res., 1977, 37: 4016-4023; Hait et al., Nature, 1976, 259: 321-323. PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Id. Inhibition of PDE4 activity results in increased cAMP levels leading to the modulation of lipopolysaccharide (LPS) induced cytokines including inhibition of TNF-α production in monocytes as well as in lymphocytes. Because PDE4 is expressed in lymphoid cells, PDE4 inhibitors have been identified as a target for the treatment of leukemias such as CLL and ALL. See, e.g., Moon & Lerner, Blood, 2003, 101(10): 4122-30; Ogawa et al., Blood, 2002, 99: 3390; U.S. Pat. No. 6,399,649; U.S. Patent Publication No. 2003/0018014.

The incidence of cancer continues to climb as the general population ages, as new cancers develop, and as susceptible populations (e.g., people infected with AIDS or excessively exposed to sunlight) grow. In particular, chronic lymphocytic leukemia is an incurable leukemia with limited therapeutic options for patients with relapsed or refractory disease. A tremendous demand therefore exists for new methods and compositions that can be used to treat patients with cancer including leukemia.

### 2.2 Methods of Treatment

Current cancer therapy may involve surgery, chemotherapy, hormonal therapy and/or radiation treatment to eradicate neoplastic cells in a patient (see, for example, Stockdale, 1998, Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Biological therapies and immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A majority of cancer chemotherapeutics act by inhibiting DNA synthesis, either directly, or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols.

Still, there is a significant need for safe and effective methods of treating, preventing and managing cancer, particularly for diseases that are refractory to standard treatments, such as surgery, radiation therapy, chemotherapy and hormonal therapy, while reducing or avoiding the toxicities and/or side effects associated with the conventional therapies.

### 2.3 PDE4 Inhibitors

Compounds referred to as PDE4 inhibitors have been synthesized and tested. These compounds potently inhibit TNF-α production, but exhibit modest inhibitory effects on LPS induced IL1β and IL12, and do not inhibit IL6 even at high drug concentrations. In addition, PDE4 inhibitors tend to produce a modest IL10 stimulation. L. G. Corral, et al., Ann. Rheum. Dis., 58:(Suppl I) 1107-1113 (1999).

PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Id. Inhibition of PDE4 activity results in increased cAMP levels leading to the modulation of LPS induced cytokines including inhibition of TNF-α production in monocytes as well as in lymphocytes.

PDE4 receptor targets have been identified in CLL cells, and inhibition of PDE4 results in CLL cell apoptosis. See Lerner & Kim, Blood, 1998, 92: 2498-2494; Moon & Lerner, Blood, 2003, 101(10): 4122-4130; U.S. Pat. No. 6,399,649; U.S. Patent Publication No. 2003/0018014. For example, Lerner and Kim have shown that rolipram, a small molecule PDE4 inhibitor, induces apoptosis in CLL cells, but not in peripheral blood whole mononuclear cells, a predominantly T-cell population. Lerner & Kim, Blood, 1998, 92: 2498-2494.

PDE4 receptor targets have also been identified in ALL cells. See Lerner & Epstein, Biochem. 1, 2006, 393: 21-42. For example, PDE4 inhibitors have been shown to induce apoptosis in human ALL cells. Ogawa et al., Blood, 2002, 99: 3390-3397. Glucocorticoids are currently the standard chemotherapeutic drugs used to treat ALL. Id. A synergistic effect was discovered when the PDE4-specific inhibitor rolipram was administered in combination with dexamethasone, a glucocorticoid. Id.

PDE4 receptor targets have been identified in DLBCL cells. For example, PDE4B is expressed in malignant B-lymphocytes and PDE4B2 expression has been shown to correlate with treatment-resistant DLBCL and relapse in DLBCL patients treated with traditional chemotherapy regimens. Smith et al., Blood, 2005, 105: 308-316.

Combination thereapy comprising a PDE4 inhibitor and a second active agent may be used to treat leukemias, including CLL and ALL. For example, the combination of rolipram and fludarabine provides a greater apoptotic effect in CLL cells than seen with either agent alone. Sigmund et al., Leukemia, 2001, 15: 1564-1571. Combination therapy targeting PDE3 as well as PDE4 has been shown to augment apoptosis in leukemic cells. The PDE3 inhibitor cilostamide has no apoptotic effects when used alone, however, CLL patients treated with cilostamide in combination with rolipram were more likely to than those treated with rolipram only. Moon et al., Clin. Cancer Res., 2002, 8: 589-595. Glucocorticoids have been shown to induce apoptosis in leukemia cells, including CLL cells. See Tsukada et al., Blood, 2001, 98: 40b-41b; Cabrelle et al., Blood, 2002, 100: 4974; Tsukada et al., Blood, 2002, 100: 3166. The combination of either rolipram or RO20-1724, each a PDE4 inhibitor, and a glucocorticoid, specifically hydrocortisone or dexamethosone, induced high levels of apoptosis in primary B-CLL cells. U.S. patent publication no. 2008/0051379. PDE7B is overexpressed in CLL cells as compared to normal B cells, and CLL cell apoptosis can also be induced by PDE7 inhibitors. The inhibition of PDE7 with selective inhibitors (BRL-50481 or IR-202) induced CLL apoptosis to a degree less than that induced by the PDE4 inhibitor rolipram, however, the dual PDE4/7 inhibitor IR-284 induced more apoptosis than either the PDE4 or PDE7 inhibitors alone. See Zhang et al., PNAS, 2008, 105(49): 19532-19537.

The combination of the PDE4-specific inhibitor rolipram and dexamethasone, a glucocorticoid, was found to have a synergistic effect in inducing apoptosis in human ALL cells. Ogawa et al., Blood, 2002, 99: 3390-3397.

Combination thereapy comprising a PDE4 inhibitor and a second active agent may be used to treat lymphomas, including diffuse large B-cell lymphoma. For example, peripheral B-cell lymphomas may be treated with a PDE4 inhibitor and chemotherapy such as chlorambucil and/or fludarabine, with antibodies such as Alemtuzumab and/or Rituximab, and/or with stem cell transplantation. See U.S. patent publication no. 2008/0051379. It was recently demonstrated that suppression of PDE4B gene expression, in combination with a pharmacological inhibitor of Syk kinase, resulted in significantly improved efficacy against DLBCL tumors. See Kim et al., Blood, 2009, 113: 6153-6160. Therefore, the combination of a PDE4 inhibitor with a Syk kinase inhibitor would also have significantly improved efficacy than either agent by itself.

Thus, inhibitors of PDE4 are promising as active agents for the treatment of leukemias and lymphomas, particularly, in CLL, ALL and DLBCL, alone or in combination with other active agents.

### 3. SUMMARY OF THE INVENTION

This invention encompasses methods of treating and preventing certain types of cancer, including primary and metastatic cancer, as well as cancers that are refractory or resistant to conventional chemotherapy. In particular, methods of this invention encompass methods of treating, preventing or managing various forms of leukemias such as chronic lymphocytic leukemia, chronic myelocytic leukemia, acute lymphoblastic leukemia, acute myelogenous leukemia and acute myeloblastic leukemia, including leukemias that are relapsed, refractory or resistant.

The methods comprise administering to a patient having cancer a therapeutically or prophylactically effective amount of a PDE4 inhibitor, or a pharmaceutically acceptable salt, solvate, hydrate, stereoisomer, clathrate, or prodrug thereof. The invention also encompasses methods of managing certain cancers (e.g., preventing or prolonging their recurrence, or lengthening the time of remission) which comprise administering to a patient having cancer a therapeutically or prophylactically effective amount of a PDE4 inhibitor of the invention, or a pharmaceutically acceptable salt, solvate, hydrate, stereoisomer, clathrate, or prodrug thereof.

In one embodiment, the PDE4 inhibitor is cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

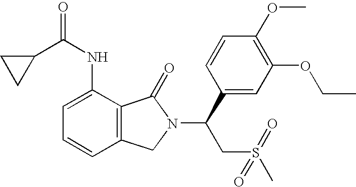

or a pharmaceutically acceptable salt or solvate (e.g., hydrate) thereof.

In one embodiment, the PDE4 inhibitor is (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

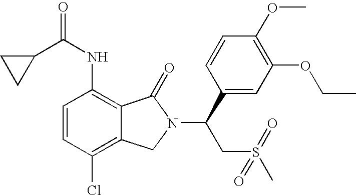

or a pharmaceutically acceptable salt or solvate (e.g., hydrate) thereof.

In one embodiment, the PDE4 inhibitor is (S)—N-{2-[(3-ethoxy-4-methoxy-phenyl)-2-methanesulfonylethyl]-1,3-dioxo-2,3-dihydro-1H-isoindol-4-yl}acetamide, which has the following chemical structure:

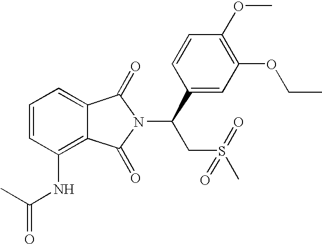

or a pharmaceutically acceptable salt or solvate (e.g., hydrate) thereof.

In one embodiment, the PDE4 inhibitor is 3-(3-acetoamidophthalimido)-3-(3-ethoxy-4-methoxyphenyl)-N-hydroxypropionamide, which has the following structure:

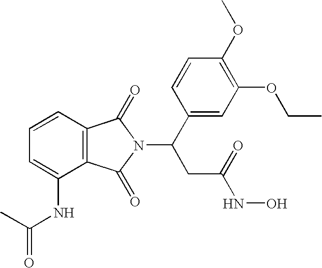

or a pharmaceutically acceptable salt or solvate (e.g., hydrate) thereof.

In one embodiment, provided herein is a method for treating, preventing and/or managing leukemia, comprising administering to a patient having leukemia a therapeutically effective amount of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

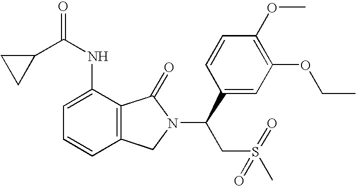

or a pharmaceutically acceptable salt or solvate (e.g., hydrate) thereof.

In another embodiment, the method for treating, preventing and/or managing leukemia, comprises administering to a patient having leukemia a therapeutically effective amount of (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

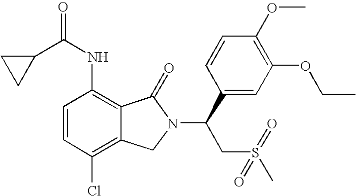

or a pharmaceutically acceptable salt or solvate (e.g., hydrate) thereof.

In another embodiment, the method for treating, preventing and/or managing leukemia, comprises administering to a patient having leukemia a therapeutically effective amount of (S)—N-{2-[1-(3-ethoxy-4-methoxy-phenyl)-2-methanesulfonylethyl]-1,3-dioxo-2,3-dihydro-1H-isoindol-4-yl}acetamide, which has the following chemical structure:

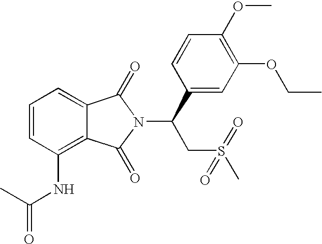

or a pharmaceutically acceptable salt or solvate (e.g., hydrate) thereof.

In another embodiment, the method for treating, preventing and/or managing leukemia, comprises administering to a patient having leukemia a therapeutically effective amount of 3-(3-acetoamidophthalimido)-3-(3-ethoxy-4-methoxyphenyl)-N-hydroxypropionamide, which has the following structure:

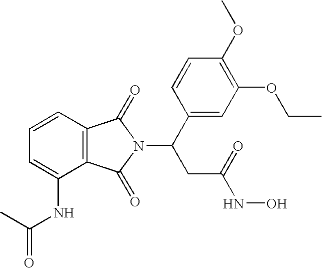

or a pharmaceutically acceptable salt or solvate (e.g., hydrate) thereof.

In one embodiment, the leukemia is chronic lymphocytic leukemia.

In one embodiment, the leukemia is acute lymphocytic leukemia.

In one embodiment, provided herein is a method for treating, preventing and/or managing lymphoma, comprising administering to a patient in need of such treatment or management a therapeutically effective amount of a PDE4 inhibitor, or a pharmaceutically acceptable salt, solvate, hydrate, stereoisomer, clathrate, or prodrug thereof.

In one embodiment, provided herein is a method for treating, preventing and/or managing of lymphoma, comprising administering to a patient having lymphoma a therapeutically effective amount of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

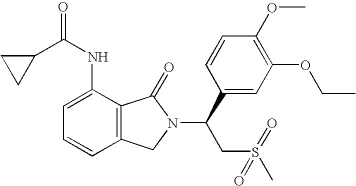

or a pharmaceutically acceptable salt or solvate (e.g., hydrate) thereof.

In one embodiment, provided herein is a method for treating, preventing and/or managing of lymphoma, comprising administering to a patient having lymphoma a therapeutically effective amount of (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

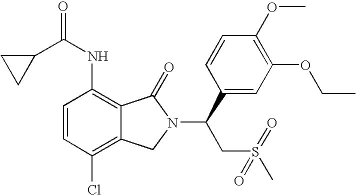

or a pharmaceutically acceptable salt or solvate (e.g., hydrate) thereof.

In another embodiment, the method for treating, preventing and/or managing lymphoma, comprises administering to a patient having lymphoma a therapeutically effective amount of (S)—N-{2-[1-(3-ethoxy-4-methoxy-phenyl)-2-methanesulfonylethyl]-1,3-dioxo-2,3-dihydro-1H-isoindol-4-yl}acetamide, which has the following chemical structure:

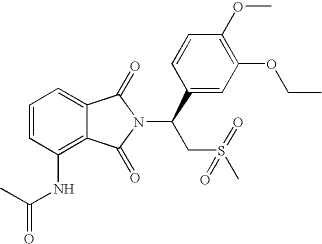

or a pharmaceutically acceptable salt or solvate (e.g., hydrate) thereof.

In another embodiment, the method for treating, preventing and/or managing lymphoma, comprises administering to a patient having lymphoma a therapeutically effective amount of 3-(3-acetoamidophthalimido)-3-(3-ethoxy-4-methoxyphenyl)-N-hydroxypropionamide, which has the following structure:

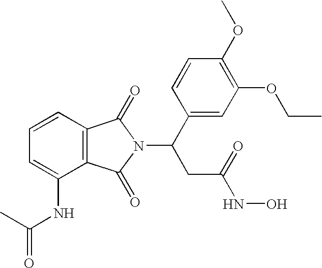

or a pharmaceutically acceptable salt or solvate (e.g., hydrate) thereof.

In one embodiment, the lymphoma is diffuse large B-cell lymphoma.

In particular methods of the invention, a PDE4 inhibitor is administered in combination with a therapy conventionally used to treat, prevent or manage cancer. Examples of such conventional therapies include, but are not limited to, surgery, chemotherapy, radiation therapy, hormonal therapy, biological therapy and immunotherapy.

This invention encompasses pharmaceutical compositions, single unit dosage forms, dosing regimens and kits which comprise a PDE4 inhibitor, or a pharmaceutically acceptable salt, solvate, hydrate, stereoisomer, clathrate, or prodrug thereof, and a second, or additional, active agent. Second active agents include specific combinations, or “cocktails,” of drugs.

### 4. BRIEF DESCRIPTION OF THE FIGURES

FIG. 1: Cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide (Compound 1) was found to increase apoptosis from 40% to 80% in CLL cells in vitro, as compared to untreated CLL cells.

FIG. 2: Cyclopropanecarboxylic acid N-{7-chloro-2-[(1S)-1-(3-ethoxy-4-methoxy-phenyl)-2-methanesulfonyl-ethyl]-3-oxo-2,3-dihydro-1H-isoindolin-4-yl}-amide (Compound 2) was found to increase apoptosis from 40% to 80% in CLL cells in vitro, as compared to untreated CLL cells.

### 5. DETAILED DESCRIPTION OF THE INVENTION

A first embodiment of the invention encompasses methods of treating, managing, or preventing cancer which comprises administering to a patient in need of such treatment or prevention a therapeutically or prophylactically effective amount of a PDE4 inhibitor of the invention, or a pharmaceutically acceptable salt, solvate, hydrate, stereoisomer, clathrate, or prodrug thereof.

In one embodiment, provided herein is a method for treating, preventing and/or managing leukemia, comprising administering to a patient in need of such treatment or management a therapeutically effective amount of a PDE4 inhibitor, or a pharmaceutically acceptable salt, solvate, hydrate, stereoisomer, clathrate, or prodrug thereof.

In one embodiment, the PDE4 inhibitor is cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

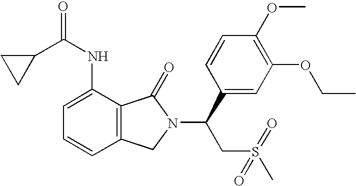

or a pharmaceutically acceptable salt or solvate (e.g., hydrate) thereof.

In one embodiment, the PDE4 inhibitor is (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

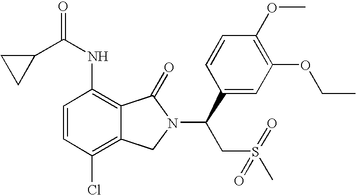

or a pharmaceutically acceptable salt or solvate (e.g., hydrate) thereof.

In one embodiment, the PDE4 inhibitor is (S)—N-{2-[1-(3-ethoxy-4-methoxy-phenyl)-2-methanesulfonylethyl]-1,3-dioxo-2,3-dihydro-1H-isoindol-4-yl}acetamide, which has the following chemical structure:

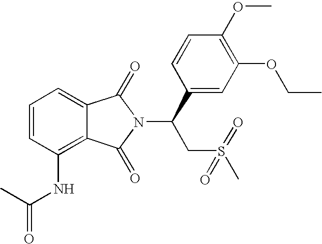

or a pharmaceutically acceptable salt or solvate (e.g., hydrate) thereof.

In one embodiment, the PDE4 inhibitor is 3-(3-acetoamidophthalimido)-3-(3-ethoxy-4-methoxyphenyl)-N-hydroxypropionamide, which has the following structure:

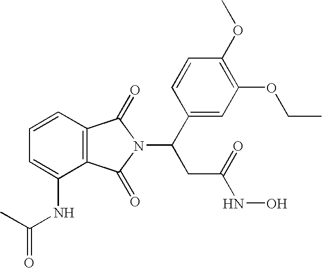

or a pharmaceutically acceptable salt or solvate (e.g., hydrate) thereof.

In one embodiment, provided herein is a method for treating, preventing and/or managing of leukemia, comprising administering to a patient having leukemia a therapeutically effective amount of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

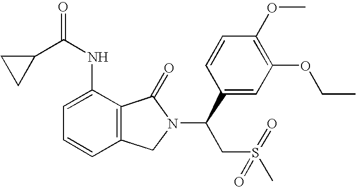

or a pharmaceutically acceptable salt or solvate (e.g., hydrate) thereof.

In another embodiment, the method for treating, preventing and/or managing leukemia, comprises administering to a patient having leukemia a therapeutically effective amount of (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

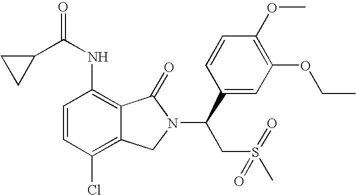

or a pharmaceutically acceptable salt or solvate (e.g., hydrate) thereof.

In another embodiment, the method for treating, preventing and/or managing leukemia, comprises administering to a patient having leukemia a therapeutically effective amount of (S)—N-{2-[1-(3-ethoxy-4-methoxy-phenyl)-2-methanesulfonylethyl]-1,3-dioxo-2,3-dihydro-1H-isoindol-4-yl}acetamide, which has the following chemical structure:

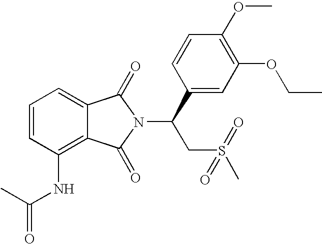

or a pharmaceutically acceptable salt or solvate (e.g., hydrate) thereof.

In another embodiment, the method for treating, preventing and/or managing leukemia, comprises administering to a patient having leukemia a therapeutically effective amount of 3-(3-acetoamidophthalimido)-3-(3-ethoxy-4-methoxyphenyl)-N-hydroxypropionamide, which has the following structure:

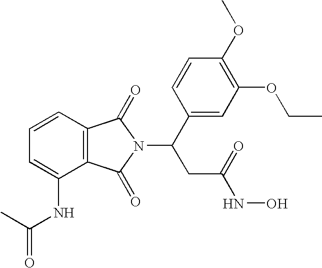

or a pharmaceutically acceptable salt or solvate (e.g., hydrate) thereof.

In one embodiment, the leukemia is chronic lymphocytic leukemia.

In another embodiment, the leukemia is acute lymphoblastic leukemia.

In certain embodiments, the methods for treating, preventing and/or managing leukemias provided herein may be used in patients that have not responded to standard treatment. In one embodiment, the leukemia is relapsed, refractory or resistant to conventional therapy.

In other embodiments, the methods for treating, preventing and/or managing leukemias provided herein may be used in treatment naive patients, i.e., patients that have not yet received treatment.

In one embodiment, provided herein is a method for treating, preventing and/or managing lymphoma, comprising administering to a patient having lymphoma a therapeutically effective amount of a PDE4 inhibitor, or a pharmaceutically acceptable salt, solvate, hydrate, stereoisomer, clathrate, or prodrug thereof.

In one embodiment, provided herein is a method for treating, preventing and/or managing of lymphoma, comprising administering to a patient having lymphoma a therapeutically effective amount of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

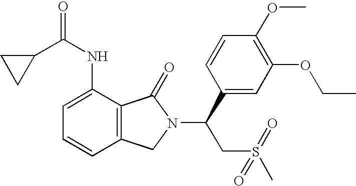

or a pharmaceutically acceptable salt or solvate (e.g., hydrate) thereof.

In another embodiment, the method for treating, preventing and/or managing lymphoma, comprises administering to a patient having lymphoma a therapeutically effective amount of (1S)-cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, which has the following structure:

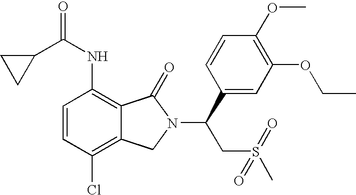

or a pharmaceutically acceptable salt or solvate (e.g., hydrate) thereof.

In another embodiment, the method for treating, preventing and/or managing lymphoma, comprises administering to a patient having lymphoma a therapeutically effective amount of (S)—N-{2-[1-(3-ethoxy-4-methoxy-phenyl)-2-methanesulfonylethyl]-1,3-dioxo-2,3-dihydro-1H-isoindol-4-yl}acetamide, which has the following chemical structure:

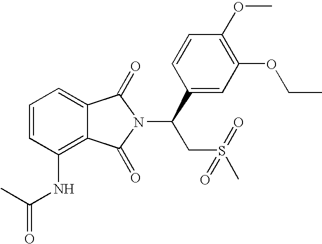

or a pharmaceutically acceptable salt or solvate (e.g., hydrate) thereof.

In another embodiment, the method for treating, preventing and/or managing lymphoma, comprises administering to a patient having lymphoma a therapeutically effective amount of 3-(3-acetoamidophthalimido)-3-(3-ethoxy-4-methoxyphenyl)-N-hydroxypropionamide, which has the following structure:

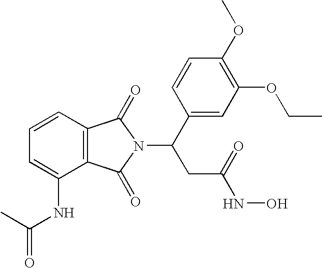

or a pharmaceutically acceptable salt or solvate (e.g., hydrate) thereof.

In one embodiment, the lymphoma is diffuse large B-cell lymphoma.

In some embodiments, the methods for treating, preventing and/or managing lymphomas provided herein may be used in patients that have not responded to standard treatment. In one embodiment, the lymphoma is relapsed, refractory or resistant to conventional therapy.

In other embodiments, the methods for treating, preventing and/or managing lymphomas provided herein may be used in treatment naive patients, i.e., patients that have not yet received treatment.

In particular methods encompassed by this embodiment, the PDE4 inhibitor is administered in combination with another drug (“second active agent”) for treating, managing, or preventing cancer. Second active agents include small molecules and large molecules (e.g., proteins and antibodies), examples of which are provided herein, as well as stem cells. Methods, or therapies, that can be used in combination with the administration of the PDE4 inhibitor include, but are not limited to, surgery, blood transfusions, immunotherapy, biological therapy, radiation therapy, and other non-drug based therapies presently used to treat, prevent or manage cancer.

In some embodiments, the PDE4 inhibitor, or a pharmaceutically acceptable salt, solvate, hydrate, stereoisomer, clathrate, or prodrug thereof is administered in combination or alternation with a therapeutically effective amount of one or more additional active agents. In one embodiment, the additional active agent is selected from the group consisting of an alkylating agent, an adenosine analog, a glucocorticoid, a kinase inhibitor, a SYK inhibitor, a PDE3 inhibitor, a PDE7 inhibitor, doxorubicin, chlorambucil, vincristine, bendamustine, forskolin, rituximab, or a combination thereof.

In one embodiment, the additional active agent is a PDE3 inhibitor.

In one embodiment, the additional active agents are a PDE3 inhibitor and a PDE7 inhibitor.

In one embodiment, the additional active agents are a cilostamide and a PDE7 inhibitor.

In one embodiment, the alkylating agent is chlorambucil.

In one embodiment, the adenosine analog is fludarabine.

In one embodiment, the PDE3 inhibitor is cilostamide.

In one embodiment, additional active agent is doxorubicin.

In one embodiment, additional active agent is forskolin.

In one embodiment, the additional active agent is rituximab.

In one embodiment, the glucocorticoid is betamethasone, budesonide, cortisone, cortisone acetate, dexamethasone, hydrocortisone, methylprednisolone, prednisolone, prednisone, triamcinolone, methylprednisolone, triamcinolone, beclomethasone, fludrocortisone acetate, deoxycorticosterone acetate (DOCA) or aldosterone.

In one embodiment, the glucocorticoid is hydrocortisone or dexamethosone.

In one embodiment, the PDE4 inhibitor is enantiomerically pure.

In one embodiment, the PDE4 inhibitor is administered in an amount of from about 1 to about 1,000 mg per day.

In one embodiment, the PDE4 inhibitor is administered in an amount of about 10, 20, 25, 50, 100, 200 or 300 mg per day.

In another embodiment, 10, 20, 25, 50, 100, 200 or 300 mg of the PDE4 inhibitor is administered twice per day.

In one embodiment, the PDE4 inhibitor is orally administered.

In one embodiment, the PDE4 inhibitor is administered in a capsule or tablet.

In one embodiment, the PDE4 inhibitor is administered in 50 mg or 100 mg of a capsule.

In one embodiment, the PDE4 inhibitor is administered for 21 days followed by seven days rest in a 28 day cycle.

The invention also encompasses pharmaceutical compositions (e.g., single unit dosage forms) that can be used in methods disclosed herein. Particular pharmaceutical compositions comprise a PDE4 inhibitor of the invention, or a pharmaceutically acceptable salt, solvate, hydrate, stereoisomer, clathrate, or prodrug thereof, and a second active agent.

### 5.1 PDE4 Inhibitors

Compounds used in the invention include racemic, stereomerically pure or stereomerically enriched PDE4 inhibitors, stereomerically or enantiomerically pure compounds that have selective cytokine inhibitory activities, and pharmaceutically acceptable salts, solvates, hydrates, stereoisomers, clathrates, and prodrugs thereof. Preferred compounds used in the invention are also known Selective Cytokine Inhibitory Drugs (SelCIDs™) of Celgene Corporation.

As used herein and unless otherwise indicated, the terms “PDE4 inhibitor,” “selective cytokine inhibitory drug” and “SelCIDs™” encompass small molecule drugs, e.g., small organic molecules which are not peptides, proteins, nucleic acids, oligosaccharides or other macromolecules. Preferred compounds inhibit TNF-α production. Further, the compounds may also have a modest inhibitory effect on LPS induced IL113 and IL12. More preferably, the compounds of the invention are potent PDE4 inhibitors. PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Without being limited by theory, inhibition of PDE4 activity results in increased cAMP levels leading to the modulation of LPS induced cytokines, including inhibition of TNF-α production in monocytes as well as in lymphocytes.

Specific examples of PDE4 inhibitors include, but are not limited to, the compounds disclosed in U.S. Pat. No. 6,667,316 and U.S. provisional patent application Nos. 60/851,152 and 60/998,716; the cyclic imides disclosed in U.S. Pat. No. 5,605,914; the cycloalkyl amides and cycloalkyl nitriles of U.S. Pat. Nos. 5,728,844 and 5,728,845, respectively; the aryl amides (for example, an embodiment being N-benzoyl-3-amino-3-(3′,4′-dimethoxyphenyl)-propanamide) of U.S. Pat. Nos. 5,801,195 and 5,736,570; the imide/amide ethers and alcohols (for example, 3-phthalimido-3-(3′,4′-dimethoxypheryl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in WO 99/06041 and substituted phenethylsulfones disclosed in U.S. Pat. No. 6,020,358; and aryl amides such as N-benzoyl-3-amino-3-(3′,4′-dimethoxyphenyl)propanamide as described in U.S. Pat. No. 6,046,221. The entireties of each of the patents and patent applications identified herein are incorporated herein by reference.

Specific PDE4 inhibitors used in the invention are cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide, cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide, N-{2-[1-(3-ethoxy-4-methoxy-phenyl)-2-methanesulfonylethyl]-1,3-dioxo-2,3-dihydro-1H-isoindol-4-yl}acetamide, 3-(3-Acetoamidophthalimido)-3-(3-ethoxy-4-methoxyphenyl)-N-hydroxypropionamide, and stereoisomers, pharmaceutically acceptable salts or solvates (e.g., hydrates) thereof.

Cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide has the following chemical structure:

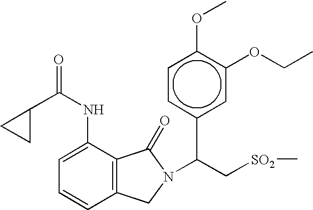

In one embodiment, the PDE4 inhibitor is cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure:

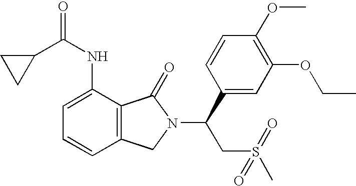

or a pharmaceutically acceptable salt or solvate (e.g., hydrate) thereof.

(1S)-Cyclopropancecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide has the following chemical structure:

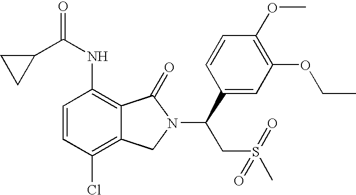

(S)—N-{2-[1-(3-ethoxy-4-methoxy-phenyl)-2-methanesulfonylethyl]-1,3-dioxo-2,3-dihydro-1H-isoindol-4-yl}acetamide (Apremilast), described in Man et al. J. Med. Chem., 2009, 52, 1522-1524, has the following chemical structure:

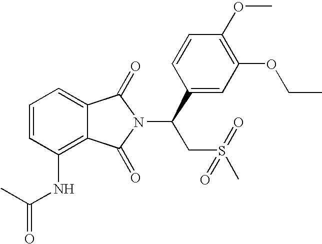

3-(3-Acetoamidophthalimido)-3-(3-ethoxy-4-methoxyphenyl)-N-hydroxypropionamide has the following structure:

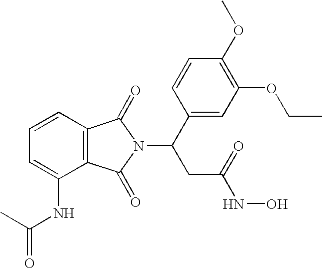

Compounds of the invention can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of PDE4 inhibitors of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of PDE4 inhibitors of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

Various PDE4 inhibitors of the invention contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of particular PDE4 inhibitors of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.2 Second Active Agents

PDE4 inhibitors can be combined with other pharmacologically active compounds (“second active agents”) in methods and compositions of the invention. It is believed that certain combinations work synergistically in the treatment of particular types of cancer. PDE4 inhibitors can also work to alleviate adverse effects associated with certain second active agents, and some second active agents can be used to alleviate adverse effects associated with PDE4 inhibitors.

One or more second active ingredients or agents can be used in the methods and compositions of the invention together with a PDE4 inhibitor. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions of the invention include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

This invention encompasses the use of native, naturally occurring, and recombinant proteins. The invention further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with compounds of the invention include monoclonal and polyclonal antibodies. Examples of antibodies include, but are not limited to, trastuzumab (Herceptin'), rituximab (Rituxan®), bevacizumab (Avastin™), pertuzumab (Omnitarg™), tositumomab (Bexxar®), edrecolomab (Panorex®), and G250. Compounds of the invention can also be combined with, or used in combination with, anti-TNF-α antibodies.

Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits of the invention. See, e.g., Emens, L. A., et al., Curr. Opinion Mol. Ther. 3(1):77-84 (2001).

In one embodiment of the invention, the large molecule active agent reduces, eliminates, or prevents an adverse effect associated with the administration of a PDE4 inhibitor. Depending on the particular PDE4 inhibitor and the cancer being treated, adverse effects can include, but are not limited to, drowsiness and somnolence, dizziness and orthostatic hypotension, neutropenia, infections that result from neutropenia, increased HIV-viral load, bradycardia, Stevens-Johnson Syndrome and toxic epidermal necrolysis, and seizures (e.g., grand mal convulsions). A specific adverse effect is neutropenia.

Second active agents that are small molecules can also be used to alleviate adverse effects associated with the administration of a PDE4 inhibitor. However, like some large molecules, many are believed to be capable of providing a synergistic effect when administered with (e.g., before, after or simultaneously) a PDE4 inhibitor. Examples of small molecule second active agents include, but are not limited to, anti-cancer agents, antibiotics, immunosuppressive agents, and steroids.

Examples of anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cyclosporin A; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, chlorambucil, fludarabine, dexamethasone (Decadron®), hydrocortisone, methylprednisolone, cilostamide, doxorubicin (Doxil®), forskolin, rituximab, cyclosporin A, cisplatin, vincristine, PDE7 inhibitors such as BRL-50481 and IR-202, dual PDE4/7 inhibitors such as IR-284, cilostazol, meribendan, milrinone, vesnarionone, enoximone and pimobendan, Syk inhibitors such as fostamatinib disodium (R406/R788), R343, R-112 and Excellair® (ZaBeCor Pharmaceuticals, Bala Cynwyd, Pa.).

### 5.3 Methods of Treatments and Prevention

Methods of this invention encompass methods of treating, preventing and/or managing various types of cancers. In one embodiment, provided herein are methods of treating, preventing or managing various types of leukemias such as chronic lymphocytic leukemia, chronic myelocytic leukemia, acute lymphoblastic leukemia, acute myelogenous leukemia, and acute myeloblastic leukemia.

As used herein, unless otherwise specified, the term “treating” refers to the administration of a compound of the invention or other additional active agent after the onset of symptoms of the particular cancer. As used herein, unless otherwise specified, the term “preventing” refers to the administration prior to the onset of symptoms, particularly to patients at risk of cancer, or characterized by, undesired angiogenesis. The term “prevention” includes the inhibition of a symptom of the particular cancer. Patients with familial history of cancer are preferred candidates for preventive regimens. As used herein and unless otherwise indicated, the term “managing” encompasses preventing the recurrence of the particular cancer in a patient who had suffered from it, and/or lengthening the time a patient who had suffered from the cancer remains in remission.

As used herein, the term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation; in particular, refractory to thalidomide.

The term “leukemia” or “leukemias” refers to malignant neoplasms of the blood-forming tissues. Leukemias include, but are not limited to, chronic lymphocytic leukemia, chronic myelocytic leukemia, acute lymphoblastic leukemia, acute myelogenous leukemia and acute myeloblastic leukemia. The leukemia may be relapsed, refractory or resistant to conventional therapy.

The term “lymphoma” or “lymphomas” refers to cancers that originate in the lymphatic system. Lymphomas include, but are not limited to, Hodgkin's lymphoma, non-Hodgkin's lymphoma, cutaneous T-Cell lymphoma, cutaneous B-Cell lymphoma, diffuse large B-Cell lymphoma, mantle cell lymphoma and low grade follicular lymphoma. The lymphoma may be relapsed, refractory or resistant to conventional therapy.

The term “relapsed” refers to a situation where patients who have had a remission of cancer (e.g., leukemia or lymphoma) after therapy have a return of cancer cells in the lymphatic system, blood and/or blood forming tissues (e.g., marrow), and/or a decrease in normal blood cells.

The term “refractory or resistant” refers to a circumstance where patients, even after intensive treatment, have residual cancer cells (e.g., leukemia or lymphoma cells) in their lymphatic system, blood and/or blood forming tissues (e.g., marrow).

The term “therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

This invention encompasses methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Methods encompassed by this invention comprise administering one or more PDE4 inhibitors of the invention, or a pharmaceutically acceptable salt, solvate, hydrate, stereoisomer, clathrate, or prodrug thereof, to a patient (e.g., a human) suffering, or likely to suffer, from cancer, specifically leukemias and lymphomas.

In one embodiment of the invention, the recommended daily dose range of a PDE4 inhibitor for the conditions described herein lie within the range of from about 1 mg to about 10,000 mg per day, given as a single once-a-day dose, or preferably in divided doses throughout a day. More specifically, the daily dose is administered twice daily in equally divided doses. Specifically, a daily dose range should be from about 1 mg to about 5,000 mg per day, more specifically, between about 10 mg and about 2,500 mg per day, between about 100 mg and about 800 mg per day, between about 100 mg and about 1,200 mg per day, or between about 25 mg and about 2,500 mg per day. In managing the patient, the therapy should be initiated at a lower dose, perhaps about 1 mg to about 2,500 mg, and increased if necessary up to about 200 mg to about 5,000 mg per day as either a single dose or divided doses, depending on the patient's global response. In a particular embodiment, cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide can be preferably administered in an amount of about 25, 50, 100, 200, 300, or 400, 800, or 1000 mg a day as two divided doses.

In a specific embodiment, cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide may be administered in an amount of about 200, 400, or 800 mg per day to patients with relapsed CLL, ALL or DLBCL. In a particular embodiment, cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide may be administered initially in an amount of 100 mg/day and the dose can be escalated every week to 200, 400, 800, and 1,000, mg/day. In a specific embodiment, the compound can be administered in an amount of up to about 1,000 mg/day to patients with CLL, ALL or DLBCL. In a particular embodiment, the compound can be administered in an amount of up to about 1,000 mg/day to patients with CLL, ALL or DLBCL.

In a specific embodiment, cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide may be administered to patients with CLL, ALL or DLBCL initially in an amount of 100 mg and can be escalated to 800 mg and 1000 mg daily.

### 5.3.1 Combination Therapy with a Second Active Agent

Specific methods of the invention comprise administering a PDE4 inhibitor of the invention, or a pharmaceutically acceptable salt, solvate, hydrate, stereoisomer, clathrate, or prodrug thereof, in combination with one or more second active agents, and/or in combination with radiation therapy, blood transfusions, or surgery. Examples of PDE4 inhibitors of the invention are disclosed herein (see, e.g., section 5.1). In one embodiment, the PDE4 inhibitor is cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, or a pharmaceutically acceptable salt or solvate (e.g., hydrate) thereof. Examples of second active agents are also disclosed herein (see, e.g., section 5.2).

Administration of the PDE4 inhibitors and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for a PDE4 inhibitor of the invention is oral. Preferred routes of administration for the second active agents or ingredients of the invention are known to those of ordinary skill in the art. See, e.g., Physicians' Desk Reference, 1755-1760 (56^{th} ed., 2002).

In one embodiment of the invention, the second active agent is administered orally, intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of PDE4 inhibitors of the invention and any optional additional active agents concurrently administered to the patient. In a particular embodiment, the second active agent is an alkylating agent, an adenosine analog, a glucocorticoid, a kinase inhibitor, a SYK inhibitor, a PDE3 inhibitor, a PDE7 inhibitor, doxorubicin, chlorambucil, vincristine, bendamustine, forskolin, rituximab, or a combination thereof.

### 5.3.2 Use with Transplantation Therapy

Compounds of the invention can be used to reduce the risk of Graft Versus Host Disease (GVHD). Therefore, the invention encompasses a method of treating, preventing and/or managing cancer, which comprises administering the PDE4 inhibitor of the invention, or a pharmaceutically acceptable salt, solvate, hydrate, stereoisomer, clathrate, or prodrug thereof, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of the PDE4 inhibitor of the invention and transplantation therapy provides a unique and unexpected synergism. In particular, a PDE4 inhibitor of the invention exhibits activity that may provide additive or synergistic effects when given concurrently with transplantation therapy in patients with cancer.

A PDE4 inhibitor of the invention can work in combination with transplantation therapy reducing complications associated with the invasive procedure of transplantation and risk of GVHD. This invention encompasses a method of treating, preventing and/or managing cancer which comprises administering to a patient (e.g., a human) a PDE4 inhibitor of the invention, or a pharmaceutically acceptable salt or solvate (e.g., hydrate) thereof, before, during, or after the transplantation of umbilical cord blood, placental blood, peripheral blood stem cell, hematopoietic stem cell preparation or bone marrow. Examples of stem cells suitable for use in the methods of the invention are disclosed in U.S. Pat. No. 7,498,171 and U.S. provisional patent application No. 60/372,348, filed Apr. 12, 2002 by R. Hariri et al., the entirety of which is incorporated herein by reference.

In one embodiment of this method, a PDE4 inhibitor of the invention is administered to patients with CLL, ALL or DLBCL before, during, or after the transplantation of autologous peripheral blood progenitor cell.

In another embodiment, a PDE4 inhibitor is administered to patients with relapsing CLL, ALL or DLBCL after the stem cell transplantation.

In another embodiment, a PDE4 inhibitor and prednisone are administered as maintenance therapy to patients with CLL, ALL or DLBCL following the transplantation of autologous stem cell.

In another embodiment, a PDE4 inhibitor and dexamethasone are administered as salvage therapy for low risk post transplantation to patients with CLL, ALL or DLBCL.

In another embodiment, a PDE4 inhibitor and dexamethasone are administered as maintenance therapy to patients with CLL, ALL or DLBCL following the transplantation of autologous bone marrow.

In another embodiment, a PDE4 inhibitor is administered following the administration of high dose of steroid and the transplantation of autologous stem cell to patients with chemotherapy responsive CLL, ALL or DLBCL.

In another embodiment, a PDE4 inhibitor and PEG INTRO-A are administered as maintenance therapy to patients with CLL, ALL or DLBCL following the transplantation of autologous CD34-selected peripheral stem cell.

In another embodiment, a PDE4 inhibitor is administered with post transplant consolidation chemotherapy to patients with newly diagnosed CLL, ALL or DLBCL to evaluate anti-angiogenesis.

### 5.3.3 Cycling Therapy

In certain embodiments, the prophylactic or therapeutic agents of the invention are cyclically administered to a patient. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one specific embodiment of the invention, a PDE4 inhibitor of the invention is administered daily in a single or divided doses in a four to six week cycle with a rest period of about a week or two weeks. The invention further allows the frequency, number, and length of dosing cycles to be increased. Thus, another specific embodiment of the invention encompasses the administration of a PDE4 inhibitor of the invention for more cycles than are typical when it is administered alone. In yet another specific embodiment of the invention, a PDE4 inhibitor of the invention is administered for a greater number of cycles that would typically cause dose-limiting toxicity in a patient to whom a second active ingredient is not also being administered.

In one embodiment, a PDE4 inhibitor of the invention is administered daily and continuously for three or four weeks at a dose of from about 1 to about 1,000 mg/d followed by a break of one or two weeks. In one embodiment, the PDE4 inhibitor is administered in an amount of about 1 to about 800 mg/d. Cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide is preferably administered daily and continuously at an initial dose of 1 to 5 mg/d with dose escalation (every week) by 10 to 100 mg/d to a maximum dose of 1,000 mg/d for as long as therapy is tolerated. In a particular embodiment, the compound is administered in an amount of about 400, 800, or 1,000 mg/day, preferably in an amount of about 800 mg/day for three to four weeks, followed by one week or two weeks of rest in a four or six week cycle.

In one embodiment of the invention, a PDE4 inhibitor of the invention and a second active ingredient are administered orally, with administration of a PDE4 inhibitor of the invention occurring 30 to 60 minutes prior to a second active ingredient, during a cycle of four to six weeks. In another embodiment of the invention, the combination of a PDE4 inhibitor of the invention and a second active ingredient is administered by intravenous infusion over about 90 minutes every cycle. In a specific embodiment, one cycle comprises the administration of from about 400 to about 800 mg/day of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide and from about 50 to about 200 mg/m²/day of a second active ingredient daily for 3 to 4 weeks and then one or two weeks of rest. In another specific embodiment, each cycle comprises the administration of from about 200 to about 400 mg/day of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide and from about 50 to about 200 mg/m²/day of a second active ingredient for three to four weeks followed by one or two weeks of rest. Typically, the number of cycles during which the combinatorial treatment is administered to a patient will be from about one to about 24 cycles, more typically from about two to about 16 cycles, and even more typically from about four to about eight cycles.

### 5.4 Pharmaceutical Compositions

Pharmaceutical compositions can be used in the preparation of individual, single unit dosage forms. Pharmaceutical compositions and dosage forms of the invention comprise a PDE4 inhibitor of the invention, or a pharmaceutically acceptable salt, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise the active ingredients disclosed herein (e.g., a PDE4 inhibitor and a second active agent). Examples of optional second, or additional, active ingredients are disclosed herein (see, e.g., section 5.2).

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a PDE4 inhibitor of the invention or a pharmaceutically acceptable salt, solvate, hydrate, stereoisomer, clathrate, or prodrug thereof in an amount of from about 0.10 to about 150 mg. Typical dosage forms comprise a PDE4 inhibitor of the invention or a pharmaceutically acceptable salt, solvate, hydrate, stereoisomer, clathrate, or prodrug thereof in an amount of about 0.1, 1, 2, 5, 7.5, 10, 12.5, 15, 17.5, 20, 25, 50, 100, 150 or 200 mg. In a particular embodiment, a preferred dosage form comprises cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide in an amount of about 1, 2, 5, 10, 25 or 50 or 100 mg. In a specific embodiment, a preferred dosage form comprises cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide in an amount of about 5, 10, 25 or 100 mg. Typical dosage forms comprise the second active ingredient in an amount of 1 to about 1000 mg, from about 5 to about 500 mg, from about 10 to about 350 mg, or from about 50 to about 200 mg. Of course, the specific amount of the anti-cancer drug will depend on the specific agent used, the type of cancer being treated or managed, and the amount(s) of a PDE4 inhibitor of the invention and any optional additional active agents concurrently administered to the patient.

### 5.4.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises a PDE4 inhibitor of the invention, anhydrous lactose, microcrystalline cellulose, polyvinylpyrrolidone, stearic acid, colloidal anhydrous silica, and gelatin.

### 5.4.2 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of a PDE4 inhibitor of the invention and its derivatives. See, e.g., U.S. Pat. No. 5,134,127, which is incorporated herein by reference.

### 5.4.4 Topical and Mucosal Dosage Forms

Topical and mucosal dosage forms of the invention include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.4.5 Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of a PDE4 inhibitor of the invention, or a pharmaceutically acceptable salt, solvate, hydrate, stereoisomer, prodrug, or clathrate thereof. Kits encompassed by this invention can further comprise additional active agents, including but not limited to those disclosed herein, for example, an alkylating agent (e.g., chlorambucil), an adenosine analog (e.g., fludarabine), a glucocorticoid (e.g., hydrocortisone or dexamethasone), a kinase inhibitor, a Syk inhibitor (e.g., fostamatinib disodium (R406/R788), R343 or Excellair®), a PDE3 inhibitor (e.g., cilostamide), a PDE7 inhibitor (e.g., BRL-50481, IR-202 or IR-284), doxorubicin, forskolin, cisplatin, vincristine, or combinations thereof.

Examples of additional active agents include, but are not limited to, those disclosed herein (See, e.g., section 5.2).

Kits of the invention can further comprise devices that are used to administer the active ingredients. Examples of such devices include, but are not limited to, syringes, drip bags, patches, and inhalers.

Kits of the invention can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

Certain embodiments of the invention are illustrated by the following non-limiting examples.

### 6.1 PREPARATION OF CYCLOPROPANECARBOXYLIC ACID {2-[(1S)-1-(3-ETHOXY-4-METHOXY-PHENYL)-2-METHANE-SULFONYL-ETHYL]-3-OXO-2,3-DIHYDRO-1H-ISOINDOL-4-YL}-AMIDE (COMPOUND (1))

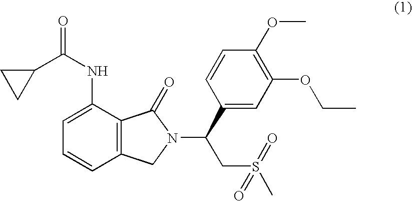

### 6.1.1 Preparation of Methyl 2-methyl-6-nitrobenzoate

A mixture of 2-methyl-6-nitrobenzoic acid (300.0 g, 1.66 moles, from Acros Organics, Morris Plains, N.J.) and trimethyl orthoacetate (298.3 g, 2.48 moles, from Aldrich Chemicals, Milwauke, Wis.) was charged into a 3-L 3-necked flask at about 20-25° C. under nitrogen. The reaction mixture was gradually heated and the low-boiling point components generated during the reaction were distilled off to an internal temperature of 95-100° C. After 2 hours, the reaction mixture was cooled to 20-25° C. over 1-2 hours. After heptane (1.50 L, from Aldrich Chemicals) was charged into the reaction mixture over 1.0-1.5 hours, the reaction mixture was seeded with methyl 2-methyl-6-nitrobenzoate (0.5 g) when it became turbid. The suspension was cooled to 0-5° C. over 0.5-1 hour and kept at 0-5° C. for another 1.5-2 hours. The solid was collected by filtration under vacuum, washed with heptane (3×300 mL), and dried to a constant weight in a tray at 30-35° C. under a vacuum at 100-120 torr. The yield of methyl 2-methyl-6-nitrobenzoate was 292.0 g (91%), based on 300.0 g of 2-methyl-6-nitrobenzoic acid. The product was found to have a purity of >99% measured by HPLC based on area percentage, and a water content of <0.1% measured by Karl Fisher titration.

### 6.1.2 Preparation of Methyl 2-bromomethyl-6-nitrobenzoate

A mixture of methyl 2-methyl-6-nitrobenzoate (200.0 g, 1.02 moles, previously prepared), 1,3-dibromo-5,5-dimethylhydantoin (DBH, 162.0 g, 0.57 mole, from Aldrich Chemicals) and methyl acetate (1.20 L, from Aldrich Chemicals) was charged into a 3-L three-necked flask at about 20-25° C. under nitrogen. After the reaction mixture was refluxed for 0.5-1 hour, a solution of 2,2′-azobisisobutyronitrile (AIBN, 8.6 g, 52 mmol, from Aldrich Chemicals) in 100 mL of methyl acetate was charged over 15-30 minutes. The reaction mixture was refluxed for 6.5-8 hours until the amount of unreacted 2-methyl-6-nitrobenzoate was less than 5-10%. The reaction mixture was cooled to 15-18° C. and kept at 15-18° C. for 50-60 minutes. The solid was filtered, washed with cold (i.e., 5-10° C.) methyl acetate (2×100 mL) until there was less than 3% of methyl 2-bromomethyl-6-nitrobenzoate remained in the solid. Next, after heptane (1.00 L) was charged into the filtrate, the upper layer organic phase was washed with 2% of brine (2×500 mL) and deionized water (1-2×500 mL) until there was less than 0.5% (area percentage at 210 nm) of unreacted 5,5-dimethylhydantoin according to measurement by HPLC. After the solution was concentrated under a reduced pressure to remove about 1.80-1.90 L of methyl acetate, methyl tert-butyl ether (MTBE, 300 mL) was charged. After the reaction mixture was refluxed at 65-70° C. for 10-15 minutes, the solution was cooled to 50-55° C. over 0.5-1 hour and seeded with 500 mg of methyl 2-bromomethyl-6-nitrobenzoate at 45-50° C. The suspension was cooled to 20-25° C. and kept at 20-25° C. for 2-3 hours. The solids were collected by filtration, washed with 5-10° C. a cold mixture of heptane and MTBE in a volume ratio of 1:2 (2×100 mL), and dried to a constant weight at 20-25° C. under a vacuum at 100-120 torr. The yield of methyl 2-bromomethyl-6-nitrobenzoate was 185.2 g (66%), based on 200.0 g input of methyl 2-methyl-6-nitrobenzoate. The product was found to have a purity of >98% measured by HPLC based on area percentage, and a water content of <0.1% measured by Karl Fisher titration.

### 6.1.3 Preparation of (1S)-1-(3-ethoxy-4-methoxyphenyl)-2-methanesulfonyl-ethylamine

After a mixture of (1S)-1-(3-ethoxy-4-methoxyphenyl)-2-methanesulfonyl-ethylamine N-acetyl-L-Leucine salt (1.10 kg, 2.46 moles), deionized water (4.40 L), and dichloromethane (DCM, 5.50 L) was charged into a reaction vessel, a solution of sodium hydroxide (196.0 g, 4.90 moles) in 1.00 L of deionized water was charged into the reaction vessel over about 5 minutes at 15-25° C. The resulting mixture was stirred for at least 10 minutes at 15-25° C. and then the aqueous and organic phases were allowed to separate. The pH of the upper aqueous phase was maintained or adjusted at pH 13-14. The phases were separated and the upper aqueous phase was extracted with DCM (2×4.4 L). The pH of the aqueous phase was maintained at 13-14 throughout the extractions. The DCM extracts were combined and washed with deionized water (3.3 L) until the pH of the aqueous phase reached 11 or less. DCM was removed under vacuum below 35° C. The water content of the residual solid should be <0.1% w/was measured by Karl Fisher titration. The residual solid was dried azeotropically with more DCM. The solid was dried to a constant weight in vacuo at 30-35° C. to give (1S)-1-(3-ethoxy-4-methoxyphenyl)-2-methanesulfonyl-ethylamine as a white powder (639.0-672.0 g, 95-100% yield).

### 6.1.4 Preparation of (1S)-7-nitro-2-[1-(3-ethoxy-4-methoxyphenyl)-2-(methylsulfonyl)ethyl]isoindolin-1-one

(1S)-7-nitro-2-[1-(3-ethoxy-4-methoxyphenyl)-2-(methylsulfonyl)ethyl]isoindolin-1-one was prepared by the following procedure. A mixture of methyl 2-bromomethyl-6-nitrobenzoate (100.0 g, 365 mmol, prepared previously in Example 5.7.2), (1S)-1-(3-ethoxy-4-methoxyphenyl)-2-methanesulfonylethylamine (104.7 g, 383 mmol, prepared previously in Example 5.7.3), sodium hydrogen carbonate (67.5 g, 8.03 moles, from Aldrich Chemicals) and dimethyl formamide (500 mL) was charged into a 1-L 3-necked flask at room temperature under nitrogen. The reaction mixture was gradually heated to an internal temperature of 70-75° C. for two hours until there was less than <2% of unreacted methyl 2-bromomethyl-6-nitrobenzoate. The reaction mixture was gradually heated to an internal temperature of 95-100° C. for 18 hours. The reaction mixture was cooled to 20-25° C. and transferred to an 1-L addition funnel. After purified water (1500 mL) was charged into a 5-L 3-necked flask, the reaction mixture in the addition funnel was added into water in the 5-L 3-necked flask at room temperature over 1-2 hours maintaining an internal temperature below 30° C. The reaction mixture was stirred for 2 hours at room temperature. The solid was filtered out under vacuum, washed with water (3×300 mL) and methanol (2×400 mL), and then charged into a 2-L 3-necked flask followed by methanol (1000 mL). The mixture was refluxed for 1 hour. The mixture was cooled to room temperature. The solid was collected by filtration under vacuum, washed with 200 mL, methanol (2 vol), and dried to a constant weight at 40-45° C. under a vacuum at 100-120 torr. The yield of (1S)-7-nitro-2-[1-(3-ethoxy-4-methoxyphenyl)-2-(methylsulfonyl)ethyl]isoindolin-1-one was 123.0 g (78%), based on 100.0 g input of methyl 2-bromomethyl-6-nitrobenzoate. The product was found to have a purity of >99% measured by HPLC based on area percentage, and a water content of <0.1% measured by Karl Fisher titration.

### 6.1.5 Alternative Preparation of (1S)-7-nitro-2-[1-(3-ethoxy-4-methoxyphenyl)-2-(methylsulfonyl)ethyl]isoindolin-1-one

(1S)-7-nitro-2-[1-(3-ethoxy-4-methoxyphenyl)-2-(methylsulfonyl)ethyl]isoindolin-1-one was also prepared by the following procedure. A mixture of methyl 2-bromomethyl-6-nitrobenzoate (100.0 g, 365 mmol, prepared previously in Example 5.7.2), (1S)-1-(3-ethoxy-4-methoxyphenyl)-2-methanesulfonyl-ethylamine (104.7 g, 383 mmol, prepared previously in Example 5.7.3), and potassium carbonate powder (100.8 g, 730 mmol, from Aldrich Chemicals) was suspended in acetonitrile (500 mL) at room temperature. The reaction mixture was refluxed at 81-83° C. for about two hours until there was less than 2% of unreacted methyl 2-bromomethyl-6-nitrobenzoate. After the reaction mixture was cooled to 45-50° C., methanol (200 mL) was charged over 5-10 minutes. After the mixture was allowed to cool to 20-25° C. and stirred for 2 hours, deionized water (1.40 L) was charged over 0.5-1 hour and stirred at 20-25° C. for 30 minutes and at 0-5° C. for 1-2 hours. The solid was filtered, washed with deionized water (3×300 mL), and dried to <10% of water content as measured by Karl Fisher titration. The solid was suspended in methanol (750 mL) and refluxed for 1-1.5 hours. The suspension was cooled to 0-5° C. over 1.5-2 hours and kept at 0-5° C. for 1-1.5 hours. The solid was filtered, washed with 0-5° C. methanol (2×200 mL) and heptane (200 mL), and then dried at 40-45° C. under vacuum to a constant weight. The yield of (1S)-7-nitro-2-[1-(3-ethoxy-4-methoxyphenyl)-2-(methylsulfonyl)ethyl]isoindolin-1-one was 148.0 g (93%), based on 100.0 g input of methyl 2-bromomethyl-6-nitrobenzoate. The product was found to have a purity of >99% measured by HPLC based on area percentage, and a water content of <1.0% measured by Karl Fisher titration.

### 6.1.6 Preparation of Compound (I)

A mixture of (1S)-7-nitro-2-[1-(3-ethoxy-4-methoxyphenyl)-2-(methylsulfonyl)ethyl]isoindolin-1-one (60 g, 138 mmol, prepared previously in Example 5.7.5), 10% Pd/C (50% wet, 2.4 g, 4 wt %, from Johnson Matthey, London, UK), ethyl acetate (780 mL) was charged into a Parr-vessel at room temperature under nitrogen. After the mixture was purged with nitrogen three times and with hydrogen three times, the reaction mixture was heated to 40° C. and then the heat was removed. The reaction mixture was stirred with hydrogen at a pressure between 40-45 psi over 4-6 hours until there was ≦3% of the hydroxylamine intermediate. The reaction mixture was cooled to 20-25° C. The reaction mixture was filtered through a celite bed (1 inch thickness) and then bed-washed with ethyl acetate (120 mL). The filtrate was transferred to a 3-L 3-necked flask equipped with a 50-mL addition funnel. After N,N-diisopropylethylamine (29 mL, 165 mmol) was charged into the flask, the addition funnel was charged with cyclopropylcarbonyl chloride (13.0 mL, 145 mmol, from Aldrich Chemicals). The cyclopropylcarbonyl chloride was added at room temperature over 1-2 hours at an internal temperature below 30° C. The reaction mixture was stirred for 2-4 hours at room temperature. After heptane (300 mL) was added, the reaction mixture was stirred for 4-6 hours. The solid was collected by filtration under vacuum, washed with 2N HCl (2×300 mL), water (2×300 mL) and then heptane (2×300 mL). The crude product was dried at 40-45° C. under a vacuum at 100-120 torr to a constant weight. The yield of crude Compound (1) was 58 g (88%), based on 60.0 g input of (1S)-7-nitro-2-[1-(3-ethoxy-4-methoxyphenyl)-2-(methylsulfonyl)ethyl]-isoindolin-1-one.

### 6.1.7 Recrystallization of Compound (1)

A mixture of crude Compound (1) (95.2 g, prepared previously in Example 5.7.6) and tetrahydrofuran (THF, 1.43 L) was charged into a 3 L flask at 20-25° C. under nitrogen. The suspension was heated to 60-65° C. until dissolution was achieved. The suspension was filtered at 45-50° C. and the solid was rinsed with 95 mL of THF prewarmed at 45-55° C. After about 950-1150 mL of THF was distilled off at normal pressure over 30-60 minutes, absolute ethanol (950 mL) was charged at 55-60° C. over 5-10 minutes. About 350-400 mL of solvents was removed at normal pressure until the internal temperature rose to 72-74° C. The resulting suspension was refluxed at 72-75° C. for 30-60 minutes, cooled to 20-25° C. over 1-2 hours and kept at 20-25° C. for another 1-2 hours. The solid was collected by filtration under vacuum, washed with absolute ethanol (240-280 mL) and heptane (240-280 mL), and then dried in tray at 50-55° C. in a vacuum at 130-140 torr to a constant weight. The yield of the off-white crystalline product was (88.0-91.0 g, 92-96%).

### 6.2 PREPARATION OF (1S)-CYCLOPROPANECARBOXYLIC ACID {7-CHLORO-2-[1-(3-ETHOXY-4-METHOXY-PHENYL)-2-METHANESULFONYL-ETHYL]-3-OXO-2,3-DIHYDRO-1H-ISOINDOL-4-YL}-AMIDE (COMPOUND (2))

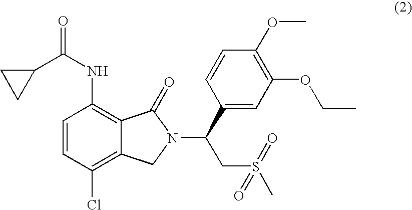

To a solution of (1S)-1-(3-ethoxy-4-methoxy-phenyl)-2-methanesulfonyl-ethylamine (1.4 g, 5.1 mmol) in DMF (20 ml) was added 2-bromomethyl-3-chloro-6-(cyclopropanecarbonyl-amino)-benzoic acid methyl ester (1.6 g, 4.6 mmol) and triethyl amine (2.0 ml, 14 mmol). The mixture was heated at 90° C. overnight. The solvent was removed in vacuo. The resulted oil was extracted with ethyl acetate (50 ml) and water (30 ml). The organic layer was washed with water (30 ml×2), brine (30 ml) and dried over magnesium sulfate. The solvent was removed in vacuo and the resulted oil was purified by silica gel column to give (1S)-cyclopropanecarboxylic acid {7-chloro-2-[1-(3-ethoxy-4-methoxy-phenyl)-2-methanesulfonyl-ethyl]-3-oxo-2,3-dihydro-1H-isoindol-4-yl}-amide as a white solid (650 mg, 30%): mp, 197-199° C.; ¹H NMR (CDCl₃) δ 0.89-0.93 (m, 2H, c-CH₂), 1.08-1.12 (m, 2H, c-CH₂), 1.44 (t, J=7.0 Hz, 3H, OCH₂CH₃), 1.65-1.69 (m, 1H, c-CH), 2.97 (s, 3H, CH₃SO₂), 3.70 (dd, J=5, 15 Hz, 1H, CHH), 3.87 (s, 3H, OCH₃), 4.05-4.23 (m, 4H, NCH+CH+OCH₂CH₃), 4.41 (d, J=17 Hz, 1H, CHH), 5.75-5.81 (m, 1H, CHN), 6.86-6.96 (m, 3H, Ar), 7.39 (d, J=9.0 Hz, 1H, Ar), 8.45 (d, J=9.0 Hz, 1H, Ar), 10.36 (s, 1H, NHCO). ¹³CNMR (CDCl₃): δ 8.5, 14.7, 16.2, 41.6, 46.9, 51.3, 55.5, 56.0, 64.7, 111.7, 112.3, 118.6, 119.3, 119.7, 121.8, 129.1, 133.2, 136.8, 139.0, 149.0, 149.9, 169.2, 172.6; Anal Calcd for C₂₄H₂₇ClN₂O₆S: C, 56.86; H, 5.37; N, 5.53. Found: C, 56.81; H, 5.26; N, 5.56.

### 6.3 PREPARATION OF (S)—N-{2-[1-(3-ETHOXY-4-METHOXY-PHENYL)-2-METHANESULFONYLETHYL]-1,3-DIOXO-2,3-DIHYDRO-1H-ISOINDOL-4-YL}ACETAMIDE (COMPOUND (3))

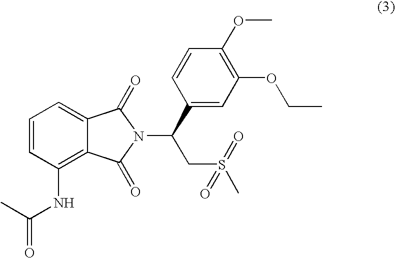

### 6.3.1 Preparation of 3-aminopthalic acid

10% Pd/C (2.5 g), 3-nitrophthalic acid (75.0 g, 355 mmol) and ethanol (1.5 L) were charged to a 2.5 L Parr hydrogenator under a nitrogen atmosphere. Hydrogen was charged to the reaction vessel for up to 55 psi. The mixture was shaken for 13 hours, maintaining hydrogen pressure between 50 and 55 psi. Hydrogen was released and the mixture was purged with nitrogen 3 times. The suspension was filtered through a celite bed and rinsed with methanol. The filtrate was concentrated in vacuo. The resulting solid was reslurried in ether and isolated by vacuum filtration. The solid was dried in vacuo to a constant weight, affording 54 g (84% yield) of 3-aminopthalic acid as a yellow product. ¹H-NMR (DMSO-d₆) δ: 3.17 (s, 2H), 6.67 (d, 1H), 6.82 (d, 1H), 7.17 (t, 1H), 8-10 (brs, 2H). ¹³C-NMR (DMSO-d₆) δ: 112.00, 115.32, 118.20, 131.28, 135.86, 148.82, 169.15, 170.09.

### 6.3.2 Preparation of 3-acetamidopthalic anhydride

A 1 L 3-necked round bottom flask was equipped with a mechanical stirrer, thermometer, and condenser and charged with 3-aminophthalic acid (108 g, 596 mmol) and acetic anhydride (550 mL). The reaction mixture was heated to reflux for 3 hours and cooled to ambient temperature and further to 0-5.degree. C. for another 1 hour. The crystalline solid was collected by vacuum filtration and washed with ether. The solid product was dried in vacuo at ambient temperature to a constant weight, giving 75 g (61% yield) of 3-acetamidopthalic anhydride as a white product. ¹H-NMR (CDCl₃) δ: 2.21 (s, 3H), 7.76 (d, 1H), 7.94 (t, 1H), 8.42 (d, 1H), 9.84 (s, 1H).

### 6.3.3 Resolution of 2-(3-ethoxy-4-methoxyphenyl)-1-(methylsulphonyl)-ethyl-2-amine

A 3 L 3-necked round bottom flask was equipped with a mechanical stirrer, thermometer, and condenser and charged with 2-(3-ethoxy-4-methoxyphenyl)-1-(methylsulphonyl)-eth-2-ylamine (137.0 g, 500 mmol), N-acetyl-L-leucine (52 g, 300 mmol), and methanol (1.0 L). The stirred slurry was heated to reflux for 1 hour. The stirred mixture was allowed to cool to ambient temperature and stirring was continued for another 3 hours at ambient temperature. The slurry was filtered and washed with methanol (250 mL). The solid was air-dried and then dried in vacuo at ambient temperature to a constant weight, giving 109.5 g (98% yield) of the crude product (85.8% ee). The crude solid (55.0 g) and methanol (440 mL) were brought to reflux for 1 hour, cooled to room temperature and stirred for an additional 3 hours at ambient temperature. The slurry was filtered and the filter cake was washed with methanol (200 mL). The solid was air-dried and then dried in vacuo at 30° C. to a constant weight, yielding 49.6 g (90% recovery) of (S)-2-(3-ethoxy-4-methoxyphenyl)-1-(methylsulphonyl)-eth-2-ylamine-N-acetyl-L-leucine salt (98.4% ee). Chiral HPLC (1/99 EtOH/20 mM KH₂PO₄ @pH 7.0, Ultron Chiral ES-OVS from Agilent Technologies, 150 mm.times.4.6 mm, 0.5 mL/min., @240 nm): 18.4 min (S-isomer, 99.2%), 25.5 min (R-isomer, 0.8%)

### 6.3.4 Preparation of Compound (3)

A 500 mL 3-necked round bottom flask was equipped with a mechanical stirrer, thermometer, and condenser. The reaction vessel was charged with (S)-2-(3-ethoxy-4-methoxyphenyl)-1-(methylsulphonyl)-eth-2-yl amine N-acetyl-L-leucine salt (25 g, 56 mmol, 98% ee), 3-acetamidophthalic anhydride (12.1 g, 58.8 mmol), and glacial acetic acid (250 mL). The mixture was refluxed over night and then cooled to <50° C. The solvent was removed in vacuo, and the residue was dissolved in ethyl acetate. The resulting solution was washed with water (250 mL×2), saturated aqueous NaHCO₃ (250 mL.times.2), brine (250 mL.times.2), and dried over sodium sulphate. The solvent was evaporated in vacuo, and the residue recrystallized from a binary solvent containing ethanol (150 mL) and acetone (75 mL). The solid was isolated by vacuum filtration and washed with ethanol (100 mL.times.2). The product was dried in vacuo at 60° C. to a constant weight, affording 19.4 g (75% yield) of Compound 3 with 98% ee. Chiral HPLC (15/85 EtOH/20 mM KH₂PO₄ @pH 3.5, Ultron Chiral ES-OVS from Agilent Technology, 150 mm×4.6 mm, 0.4 mL/min., @240 nm): 25.4 min (S-isomer, 98.7%), 29.5 min (R-isomer, 1.2%). ¹H-NMR (CDCl₃) δ: 1.47 (t, 3H), 2.26 (s, 3H), 2.87 (s, 3H), 3.68-3.75 (dd, 1H), 3.85 (s, 3H), 4.07-4.15 (q, 2H), 4.51-4.61 (dd, 1H), 5.84-5.90 (dd, 1H), 6.82-8.77 (m, 6H), 9.46 (s, 1H). ¹³C-NMR (DMSO-d₆) δ: 14.66, 24.92, 41.61, 48.53, 54.46, 55.91, 64.51, 111.44, 112.40, 115.10, 118.20, 120.28, 124.94, 129.22, 131.02, 136.09, 137.60, 148.62, 149.74, 167.46, 169.14, 169.48.

### 6.4 PREPARATION OF 3-(3-ACETOAMIDOPHTHALIMIDO)-3-(3-ETHOXY-4-METHOXYPHENYL)-N-HYDROXYPROPIONAMIDE (COMPOUND (4))

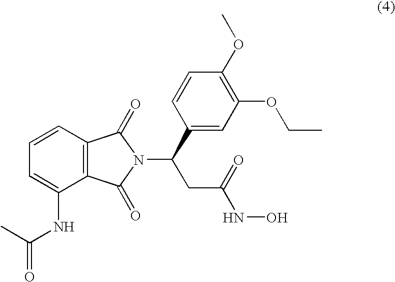

A mixture of 3-(3-acetoamidophthalimido)-3-(3-ethoxy-4-methoxyphenyl)propanoic acid (2.0 g, 4.7 mmol) and carbonyldiimidazole (1.14 g, 7.03 mmol) in THF (10 mL) under N₂ was stirred at room temperature for 2 h. To the resulting solution was added hydroxylamine hydrochloride (651 mg, 9.37 mmol). The resulting suspension was stirred for 18 h. To the suspension was added water (150 mL) and stirring was continued for 1 h. The suspension was filtered, the solid was washed with water (5×30 mL) and ether (2×20 mL), and then was dried in a vacuum oven overnight (60° C., <1 torr) to give 3-(3-acetoamidophthalimido)-3-(3-ethoxy-4-methoxyphenyl)-N-hydroxypropionamide as a white solid (1.0 g, 48% yield): mp, 117.0-119.0° C.; ¹H NMR (DMSO-d₆) δ 1.31 (t, J=6.9 Hz, 3H, CH₃), 2.19 (s, 3H, CH₃), 3.09 (d, J=7.9 Hz, 2H, CH₂), 3.72 (s, 3H, CH₃), 3.98 (q, J=7.0 Hz, 2H, CH₂), 5.65 (t, J=7.8 Hz, 1H, NCH), 6.87-6.95 (m, 2H, Ar), 6.99 (br s, 1H, Ar), 7.54 (d, J=6.9 Hz, 1H, Ar), 7.77 (t, J=7.45 Hz, 1H, Ar), 8.41-8.47 (m, 1H, Ar), 8.80 (br s, 1H, OH), 9.71 (s, 1H, NH), 10.59 (br s, 1H, NH); ¹³C NMR (DMSO-d₆) δ 24.69, 34.20, 44.09, 60.04, 65.48, 73.79, 121.78, 122.43, 126.69, 127.97, 129.64, 135.83, 141.00, 141.44, 145.75, 146.38, 157.70, 158.59, 175.97, 177.13, 178.19, 179.22; Anal Calcd for C₂₂H₂₃N₃O₇+0.3H₂O: C, 59.14; H, 5.32; N, 9.40. —Found: C, 59.32; H, 5.33; N, 9.02.

### 6.5 Inhibition of PDE4

Phosphodiesterase 4 enzyme was purified from U937 human monocytic cells by gel filtration chromatography, and phosphodiesterase reactions were carried out as previously described. See, e.g., Muller et al., Bioorg. Med. Chem. Lett., 1998, 8(19): 2669-2674. Briefly, reactions were carried out in 96-well deep-well plates in 50 mM Tris HCl pH 7.5, 5 mM MgCl₂, 1 μM cyclic adenosine monophosphate (cAMP), plus 10 nM [³H]-cAMP for 45 min at 30° C. The reactions were terminated by boiling, treated with 1 mg/ml snake venom, and separated using AG-1X8 ion exchange resin (BioRad). Reactions consumed less than 15% of available substrate. Compound 1 and Compound 2 inhibited PDE4 with an IC₅₀ of 105 nM and 7.8 nM, respectively. Compound 3 inhibited PDE4 with an IC₅₀ of 73.5 nM. Compound 4 inhibited PDE4 with an IC₅₀ of 38 nM.

### 6.6 Induction of Apoptosis in Primary CLL Cells

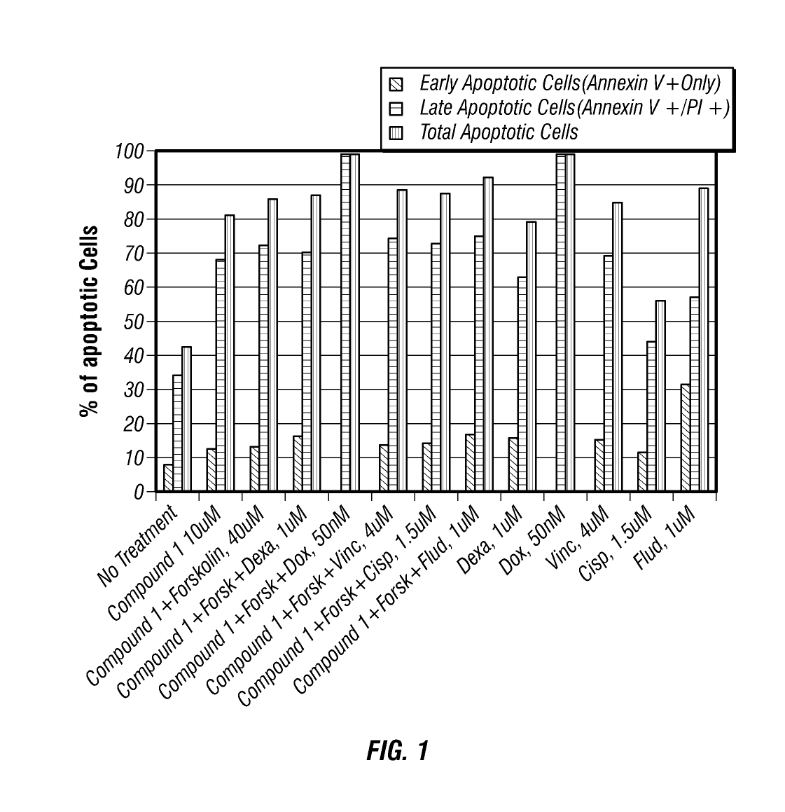

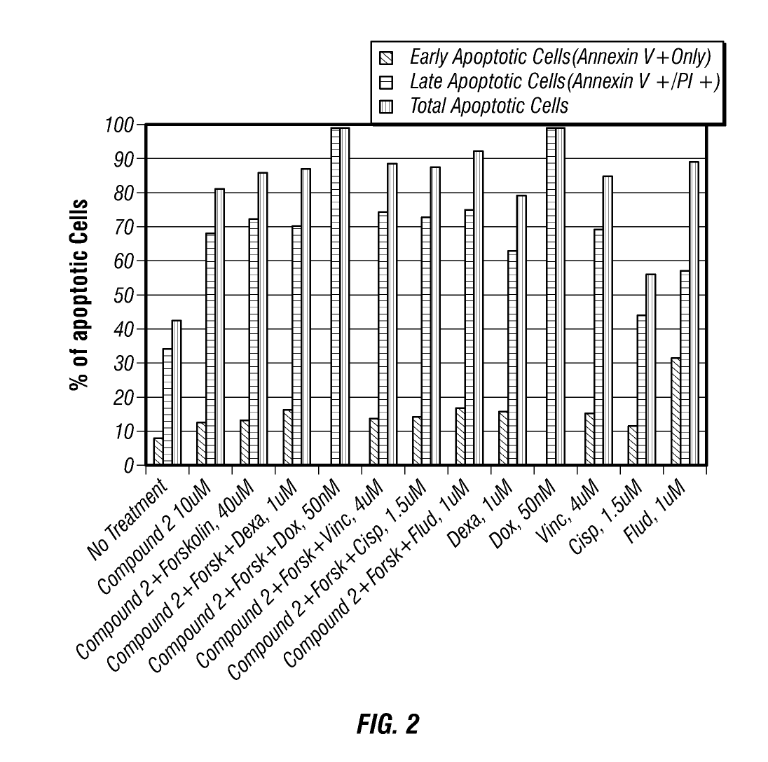

Primary CLL cells (ProteoGenex) were cultured with RPMI-1640 complete medium containing 10% FBS. Forskolin (40 μM, Sigma) was added 1 hour prior to the addition of PDE4 inhibitors (at 10 μM) and chemotherapeutic drugs such as dexamathasone, doxorubicin, vincristine, cisplatin, and fludarabine (Sigma), at a concentration of approximately half of their IC₅₀ in other cell lines. The cells were then incubation for 48 hours and analyzed for apoptotic cells by using apoptosis detection kit (APO-Direct, BD Pharmingen) as per the manufacturer's instructions. The background apoptosis from this CLL donor was 42% (FIG. 1). Forskolin at 40 μM alone did not increase the apoptosis of CLL cells. Compound 1 alone increased CLL cell apoptosis to about 80%. The chemotherapeutic drugs (except cisplatin) also increased apoptosis. However, the combination of Compound 1 with forskolin and chemotherapeutic drugs did not lead to further increased apoptosis. Compound 2 (a PDE4 inhibitor which also inhibits the multidrug resistance pumps P-gp and MRP-1) showed similar results to Compound 1 (FIG. 2). In these particular CLL cells, Pgp and MRP-1 expression was undetectable (data not shown), which would negate the potential benefit of Compound 2 vs. Compound 1.

The ability of a PDE4 inhibitor to induce apoptosis in primary chronic lymphocytic leukemia (CLL) cells is investigated in an in vitro study. CLL cells are incubated for 48 hours (i) alone; (ii) in the presence of Compound 1; (iii) in the presence of Compound 1 and forskolin; (iv) in the presence of Compound 1, forskolin and dexamethasone; (v) in the presence of Compound 1, forskolin and doxorubicin; (vi) in the presence of Compound 1, forskolin and vincristine; (vii) in the presence of Compound 1, forskolin and cisplatin; and (viii) in the presence of Compound 1, forskolin and fludarabine.

As shown in FIG. 1, Compound 1 alone was found to increase apoptosis from 40% to 80% as compared to untreated CLL cells.

### 6.7 In Vivo LPS-Induced TNF-α Production Assay

Male CD rats procured from Charles River Laboratories at seven weeks of age are allowed to acclimate for one week prior to use. A lateral tail vein is cannulated percutaneously with a 22-gage over-the-needle catheter under brief isoflurane anesthesia. Rats are administered a PDE4 inhibitor of the invention either by intravenous injection via the tail vein catheter or oral gavage 15 to 180 min prior to injection of 0.05 mg/kg LPS (E. Coli 055:B5). Catheters are flushed with 2.5 mL/kg of normal injectable saline. Blood is collected via cardiac puncture 90 minutes after LPS challenge. Plasma is prepared using lithium heparin separation tubes and frozen at −80° C. until analyzed. TNF-α levels are determined using a rat specific TNF-α ELISA kit (Busywork). The ED₅₀ values are calculated as the dose of the PDE4 inhibitor of the invention at which the TNF-α production is reduced to 50% of the control value. Compound 1 inhibited TNF-α levels in rat plasma with an approximate ED₅₀ of 0.0078 mg/kg p.o. Compound 2 inhibited TNF-α levels in rat plasma by 70% at 0.1 mg/kg p.o. and 84% at 1 mg/kg p.o.

### 6.8 Cycling Therapy in Patients

In a specific embodiment, a PDE4 inhibitor of the invention are cyclically administered to patients with cancer. Cycling therapy involves the administration of a first agent for a period of time, followed by a rest for a period of time and repeating this sequential administration. Cycling therapy can reduce the development of resistance to one or more of the therapies, avoid or reduce the side effects of one of the therapies, and/or improves the efficacy of the treatment.

In a specific embodiment, prophylactic or therapeutic agents are administered in a cycle of about four to six weeks, about once or twice every day. One cycle can comprise the administration of a therapeutic on prophylactic agent for three to four weeks and at least one week or two weeks of rest. The number of cycles administered is from about one to about 24 cycles, more typically from about two to about 16 cycles, and more typically from about four to about eight cycles.

For example, in a cycle of four weeks, on day 1, the administration of 800 mg/d of Compound 1, 2, 3 or 4 is started in patients with CLL, ALL or DLBCL. On day 22, the administration of the compound is stopped for a week of rest. On day 29, the administration of 800 mg/d of the compound is begun.

### 6.9 DLBCL Cell Proliferation Assay

Diffuse large B-cell lymphoma cell proliferation was assessed by the ³H-thymidine incorporation assay. Briefly, cells are cultured in 96-well cell culture plates in the presence or absence of drug. Each well contained 3000 cells/75 μL cell culture medium (Roswell Park Memorial Institute [RPMI]-4640+10-20% fetal bovine serum [FBS], 1% pen/strep/1% L-glutamine). Compound dilutions were made in 4× the required final concentration, and 25 μl of each compound was added to the cells in triplicate. The cells are treated with drug at 0.0001-100 μM final in a final concentration of 0.25% dimethyl sulfoxide (DMSO) for all samples.

Cells are grown at 37° C. in a humidified incubator at 5% CO, for 72 hours in the presence of the test compounds. One microcurie of ³H-thymidine (GE Healthcare, Fairfield, Conn.) is added to each well for the final 6 hours of culture. The cells are harvested onto UniFilter-96 GF/C filter plates (PerkinElmer, Waltham, Mass.) using a cell harvester (Tomtec, Hamden, Conn.), and the plates are allowed to dry overnight. A total of 25 μL/well of Microscint™-20 (PerkinElmer) is added and the plates are analyzed in TopCount NXT (PerkinElmer). Each well is counted for 1 minute. The percentage inhibition of cell proliferation is calculated by averaging all triplicates and normalizing to the DMSO control (0% inhibition). Final cumulative half-maximal inhibitory concentrations (IC₅₀) are calculated using non-linear regression and sigmoidal dose-response, constraining the top to 100% and bottom to 0% and allowing variable slope, using GraphPad Prism version 5.01. IC₅₀ for Compound 1 and Compound 3 were each >1 μM.

### 6.10 Clinical Studies in Patients with CLL

Patients with CLL are treated with up to four cycles of Compound 1, 2, 3 or 4 (about 1 to 100 mg daily, oral administration) every four to six weeks. Maintenance treatment consisting of daily Compound 1, 2, 3 or 4 is continued until disease progression. The therapy comprising the administration of Compound 1, 2, 3 or 4 is highly active and generally tolerated in CLL patients whose prognosis is otherwise poor.

Alternatively, the above treatment regimen may be modified to administer Compound 1, 2, 3 or 4 (about 1 to 100 mg daily, oral administration) in combination with chlorambucil (0.4 to 0.8 mg/kg orally on days 1 and 15 every 4 weeks), fludarabine (25 mg/m² for 5 days intravenously, every 28 days), bendamustine (100 mg/m²/d intravenously on days 1 to 2 every 4 weeks), doxorubicin (35 mg/m² as a bolus infusion or 9 mg/m² per day as a constant-rate infusion for a period of 96 h), vincristine (0.5, 1, or 1.5 mg/m²), PDE7 inhibitors (e.g., BRL-50481, IR-202), the dual PDE4/7 inhibitor IR-284, the Syk inhibitors piceatannol or fostamatinib, and/or dexamethasone (40 mg/day orally on days 1 to 4), every four to six weeks. Maintenance treatment consisting of daily a PDE4 inhibitor of the invention and monthly dexamethasone are continued until the disease progression. The therapy comprising the administration of Compound 1, 2, 3 or 4 and dexamethasone is highly active and generally tolerated in CLL patients whose prognosis is otherwise poor.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

1. A method of treating chronic lymphocytic leukemia, which comprises administering to a patient having chronic lymphocytic leukemia a therapeutically effective amount of cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide, which has the following structure: 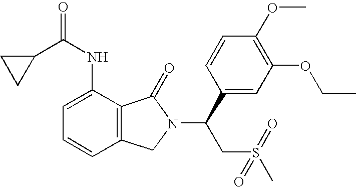
or a pharmaceutically acceptable salt or solvate thereof.

2.-12. (canceled)

13. The method of claim 1, further comprising the administration of a therapeutically effective amount of one or more additional active agents.

14. The method of claim 13, wherein the additional active agent is selected from the group consisting of an alkylating agent, an adenosine analog, a glucocorticoid, a kinase inhibitor, a SYK inhibitor, a PDE3 inhibitor, a PDE7 inhibitor, doxorubicin, chlorambucil, vincristine, bendamustine, forskolin and rituximab.

15. The method of claim 13, wherein the additional active agent is a PDE3 inhibitor.

16. The method of claim 13, wherein the additional active agents are a PDE3 inhibitor and a PDE7 inhibitor.

17. The method of claim 13, wherein the additional active agents are a cilostamide and a PDE7 inhibitor.

18. The method of claim 13, wherein the additional active agent is rituximab.

19. The method of claim 1, wherein the compound is enantiomerically pure.

20. The method of claim 1, wherein the compound is administered in an amount of from about 1 to about 1,000 mg per day.

21. The method of claim 20, wherein the compound is administered in an amount of about 10, 20, 25, 50, 100, 200 or 300 mg per day.

22. The method of claim 20, wherein the compound is orally administered.

23. The method of claim 20, wherein the compound is administered in a capsule or tablet.

24. The method of claim 23, wherein the compound is administered in 50 mg or 100 mg of a capsule.

25. The method of claim 1, wherein the chronic lymphocytic leukemia is relapsed, refractory or resistant to conventional therapy.

26. (canceled)

27. (canceled)

28. The method of claim 1, wherein the compound is administered for 21 days followed by seven days rest in a 28 day cycle.
